(19) United States
(12) Patent Application Publication
(10) Pub. No.: US 2011/0303228 A1
(43) Pub. Date: Dec. 15, 2011

(54) INCONTINENCE TREATMENT SYSTEM

(75) Inventor: Michael M. Witzmann, Minneapolis, MN (US)

(73) Assignee: COLOPLAST A/S, Humlebaek (DK)

(21) Appl. No.: 12/898,716

(22) Filed: Oct. 6, 2010

(30) Foreign Application Priority Data
Jun. 14, 2010 (DK) PA 2010 70259

(51) Int. Cl. A61F 5/48

(52) U.S. Cl. 128/885

(57) ABSTRACT

An incontinence treatment system includes a rigid implant having an aspect ratio of greater than 5 and an insertion tool. The insertion tool includes a post fixed to a handle and a sheath disposed around the post. The sheath is axially retractable relative to the post and has a length that is greater than a length of the post. The insertion tool has a loaded state in which the sheath extends distal the post to provide a recess at a distal end of the post that is sized to receive the rigid implant. The insertion tool has a deployed state in which the sheath retracts in a proximal direction toward the handle to expose the distal end of the post and allow the post to push the rigid implant out of the recess and into tissue.

### BACKGROUND

Devices for treating urinary incontinence include slings, supports, and other scaffold-like devices that are implanted in a patient's body to support the urethra.

A sub-urethral sling is a urinary incontinence treatment device that is surgically implanted under the urethra to support the urethra and inhibit urine from leaking out of the urethra during a provocative event such as coughing or sneezing. Implanting an incontinence treatment device and anatomically securing the device has the potential to be a difficult and time consuming procedure. In addition, some sub-urethral sling devices may suffer from unreliable anatomical fixation and/or unacceptable adjustment or tensioning relative to the urethra, which has the potential to produce suboptimal or even unacceptable results in the treatment of urinary incontinence.

Other urinary incontinence treatment devices, such as injected bulking liquids, are applied to coaptate the urethra. However, coaptation of the urethra can potentially erode the urethral tissue over time. Erosion of the urethra is particularly undesirable with the female urethra, which is relatively short (about 3 cm in length) as compared to the male urethra. In addition, the beneficial effects of an injected bulking agent can decrease over time if the health of the urethra, due to natural circumstances, continues to diminish.

Improved incontinence treatment methods and devices would be welcomed by both the patient and the surgical staff.

### SUMMARY

One aspect provides a method of treating incontinence that includes inserting a rigid implant peri-urethrally into a patient, aligning a longitudinal axis of the rigid implant substantially parallel to a urethra of the patient, and limiting mobility of the urethra with the rigid implant.

One aspect provides an incontinence treatment system that includes a rigid implant having an aspect ratio of greater than 5 and an insertion tool. The insertion tool includes a post fixed to a handle and a sheath disposed around the post. The sheath is axially retractable relative to the post and has a length that is greater than a length of the post. The insertion tool has a loaded state in which the sheath extends distal the post to provide a recess at a distal end of the post that is sized to receive the rigid implant. The insertion tool has a deployed state in which the sheath retracts in a proximal direction toward the handle to expose the distal end of the post and allow the post to push the rigid implant out of the recess and into tissue.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 is a side view of one embodiment of an incontinence treatment system including an insertion tool and an implant.

FIG. 2A is a side view and FIG. 2B is an end view of one embodiment of the implant illustrated in FIG. 1.

FIG. 3A is a side view and FIG. 3B is an end view of one embodiment of a tapered implant.

FIG. 4A is a side view and FIG. 4B is an end view of one embodiment of a pre-stressed implant.

FIG. 5 is a cross-sectional view of the incontinence treatment system illustrated in FIG. 1 in a loaded state.

FIG. 6 is a cross-sectional view of the incontinence treatment system illustrated in FIG. 1 in a deployed state.

FIG. 7 is a cross-sectional view of the incontinence treatment system employed to deploy an implant into tissue of the patient.

FIG. 8 is a schematic view of one embodiment of a female urethra supported by an implant.

FIG. 9 is a schematic view of one embodiment of a female urethra supported by multiple implants.

FIG. 10 is a cross-sectional view of the female urethra and the implants illustrated in FIG. 9.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

It is to be understood that the features of the various exemplary embodiments described herein may be combined with each other, unless specifically noted otherwise.

Tissue includes soft tissue, which includes dermal tissue, sub-dermal tissue, ligaments, tendons, or membranes. As employed in this specification, the term “tissue” does not include bone.

Peri-urethral means adjacent to or located near the urethra. The urethra is formed by a urethral canal that is surrounded (in order, radially away from the urethral canal) by submicosa tissue, longitudinal smooth muscle, circular smooth muscle, and striated muscle. The peri-urethral location is any of the tissues outside of the urethra.

Limiting mobility of the urethra means limiting the undesired movement of the urethra, or decreasing or eliminating hyper-mobility of the urethra.

People who are incontinent may be segregated into two groups: those with hyper-mobile urethras and those whose urethras are not hyper-mobile. A hyper-mobile urethra will translate into alignment with an exit of the bladder, thus creating a “straight-shot” pathway from the bladder that allows urine to escape from the bladder and out of the urethra. Physicians have developed an evaluation to determine if the patient has a hyper-mobile urethra. The evaluation entails the placement of an indicator stick into the longitudinal axis of the urethra such that a portion of the indicator stick extends distally out of the patient's body (those of skill in the art refer to the evaluation as the “Q-tip™ test”). The patient is prompted to initiate a provocative event, for example a cough or a tightening of the abdominal muscles, and the physician observes the indicator stick for movement. Movement of the indicator stick indicates that the longitudinal axis of the urethra is moving, which is indicative of the patient having a hyper-mobile urethra.

The urethra is normally supported by connective and other tissues. Over time, and particularly with parous women, the support of the urethra erodes, giving rise to hyper-mobility of the urethra. As described above, hyper-mobile urethras are susceptible to the undesirable leaking of urine during provocative events such as sneezing, laughing, or coughing (which is sometimes referred to as stress urinary incontinence). The implant described herein reduces or eliminates hyper-mobility of the urethra by improving the support provided to the urethra without coaptating the urethra, in a way that reduces or eliminates the undesirable leaking of urine from the urethra.

Embodiments provide an incontinence treatment system including an insertion tool and in implant. The insertion tool is employed to place the implant into tissue that is external relative to the urethra, where the implant supports the urethra to limit mobility of the urethra. Embodiments provide an implant that is configured (e.g., shaped or pre-stressed) to support the urethra when implanted into tissue remote from the urethra. Embodiments provide an implant that includes an agent or additive that is provided to induce a fibrotic response in tissue when implanted. Fibrosis of the tissue around portions of the urethra will lead to hardening of the peri-urethral tissue and reduced mobility of the urethra.

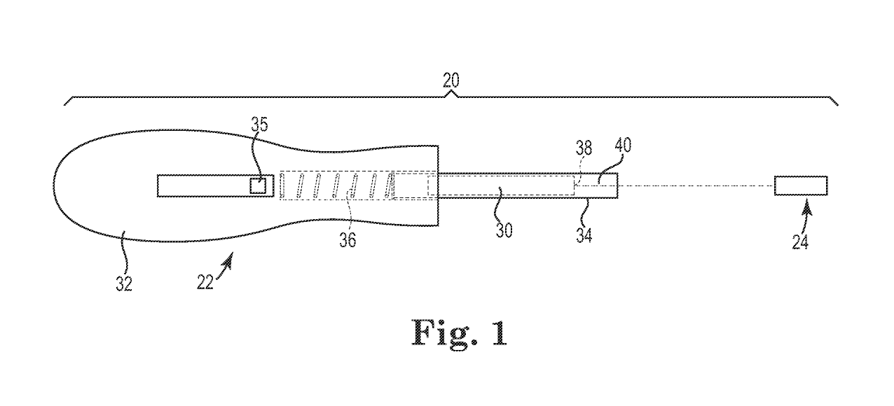

FIG. 1 is a side view of one embodiment of an incontinence treatment system 20 including an insertion tool 22 and an implant 24. The insertion tool 22 includes a post 30 fixed to a handle 32, and a sheath 34 disposed around the post 30 that is retractable into the handle 32 via a sliding button 35.

In one embodiment, the post 30 is fabricated from stainless steel and is connected to a plastic handle 32. The sheath 34 is suitably fabricated from plastic such as polyethylene, polypropylene, or polyester or from metal such as stainless steel.

In one embodiment, the sheath 34 is connected to the sliding button 35 and is biased between a loaded state and a deployed state, for example by a spring 36 disposed within the handle 32. The loaded state is characterized by the sheath 34 extending beyond a distal end 38 of the post 30 to provide a recess 40 that is sized to receive the implant 24. The deployed state is characterized by the sheath 34 being retracted into the handle 32 to expose the distal end 38 of the post 30, which allows the distal end 38 of the post 30 to push the implant 24 out of the recess 40 and into tissue.

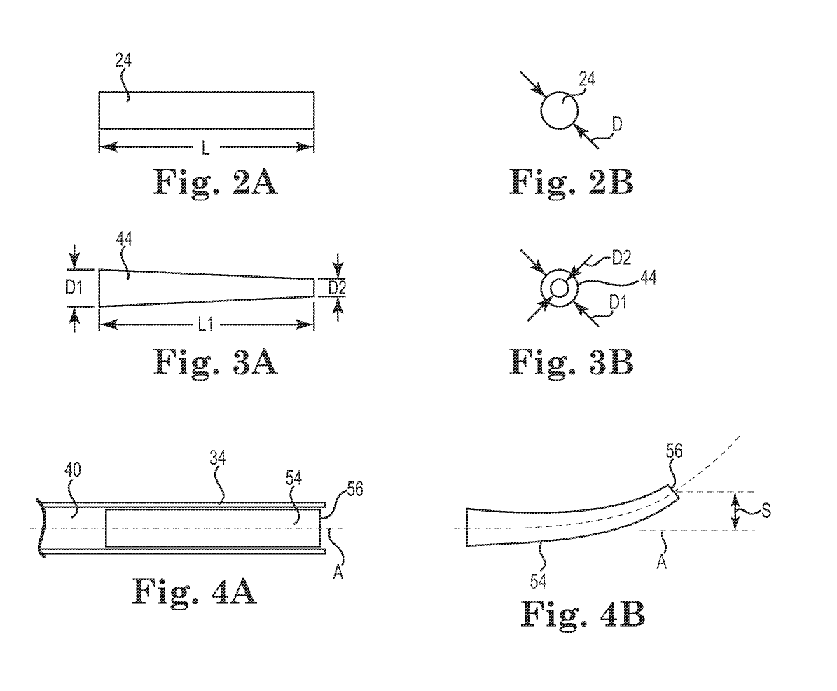

FIG. 2A is a side view and FIG. 2B is an end view of the implant 24. In one embodiment, the implant 24 is a cylindrical implant having a length L and a diameter D. In general, the length of the female urethra is approximately 3 cm, and a length L of the implant 24 is between about 1-3 cm and the D is between about 1-8 mm.

An aspect ratio of the implant is defined to be the ratio of the length L of the implant 24 to the diameter D of the implant 24 (i.e., L/D). In one embodiment, the length L of the implant 24 is 2 cm (i.e., 20 mm) and the diameter D of the implant 24 is 3 mm to provide an aspect ratio for the implant 24 of greater than 6. In another embodiment, the length L of the implant 24 is 3 cm (i.e., 30 mm) and the diameter D of the implant 24 is 2 mm to provide an aspect ratio for the implant 24 of 15, or an aspect ratio that is greater than 10.

Suitable materials for fabricating the implant 24 include polyester, silk, stainless steel, braided materials such as braided polyester strands or braided silk strands, or springs including helical springs or non-helical springs. In one embodiment, the implant 24 is provided as a rigid implant formed from multiple strands that are wound or braided into a cable having an axial stiffness configured to have a bending resistance and stiffness that is significantly greater than the stiffness of peri-urethral tissue.

In one embodiment, the implant 24 is formed from a rigid polyester material in a cylindrical shape having a length of about 2 cm and a diameter of about 3 mm to provide a foreign body that is implantable peri-urethrally into the patient and configured to induce a fibrotic response in the tissue.

A variety of means for inducing a fibrotic response in the tissue are available, including fabricating the implant 24 from a foreign body material that induces a fibrotic response, coating the implant 24 with a fibrosis-inducing agent, imbuing the implant 24 with a fibrosis-inducing agent, to name several approaches.

In one embodiment, the implant to 22 is formed from stainless steel and is treated with an agent that is configured to induce a fibrotic response in tissue when implanted in the patient. Suitable agents for creating a fibrotic response include, for example, a polyester coating deposited over the stainless steel implant, or coatings or additives of protein, peptide, collagen, or Laminan.

FIG. 3A is a side view and FIG. 3B is an end view of one embodiment of an implant 44. In one embodiment, the implant 44 is a tapered implant having a length L1 and a first diameter D1 and a second diameter D2. A suitable length L1 of the implant 44 is between about 1-3 cm. The implant 44 tapers from the first diameter D1 to the second diameter D2, where the first diameter D1 is about 3-6 mm and the second diameter D2 is about 1-2 mm. In one embodiment, the diameter D1 is about 4 mm in the diameter D2 is about 1 mm, such that the tapered implant 44 facilitates pushing the implant 44 into tissue. The implant 44 is suitably fabricated from the materials described above for implant 24

FIG. 4A is a side view of one embodiment of a pre-stressed implant 54 constrained by the sheath 34 (FIG. 1) and FIG. 4B is an end view of the pre-stressed implant 54. In one embodiment, the implant 54 has a longitudinal axis A and is fabricated from plastic or metal to include a pre-stressed bend such that one end 56 of the implant 54 diverges away from the longitudinal axis A by a distance when the implant is deployed in tissue (i.e., unconstrained by the insertion tool 22).

The pre-stressed bend of the implant 54 is constrained by the walls of the sheath 34 when the pre-stressed implant 54 is loaded into the insertion tool 22 (FIG. 1). After implantation when the implant 54 is inserted into tissue, the pre-stressed bend is unconstrained and allows the end 56 of the implant 54 to diverge away from the longitudinal axis A by the distance to deliver additional compression or tension to the tissue that acts through the peri-urethral tissue to support the urethra. The pre-stressed bend is bendable such that the implant is constrained in a linear configuration when engaged in the insertion tool 22. In one embodiment, the implant 54 is fabricated from the materials described above for the implant 24 and can include an agent or a coating that induces a fibrotic response in the tissue.

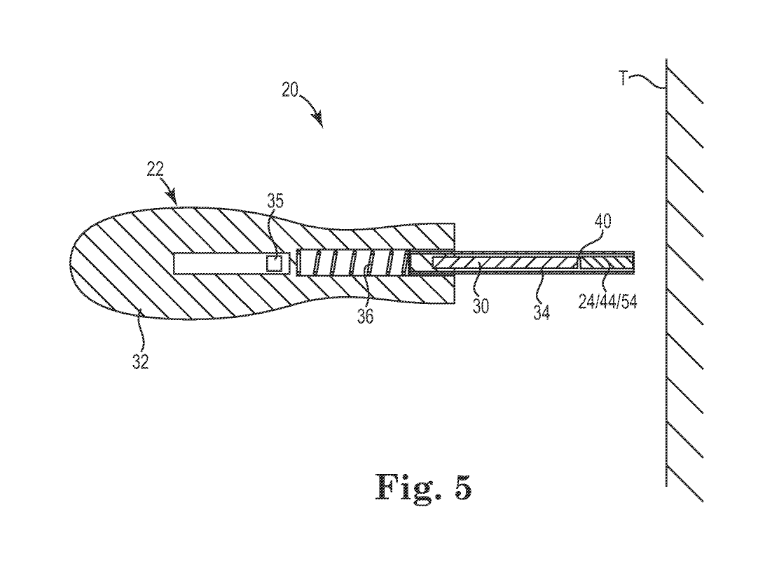

FIGS. 5-7 illustrate a method of treating incontinence through the use of the incontinence treatment system 20.

FIG. 5 is a cross-sectional view of the incontinence treatment system 20 in a loaded state. The sheath 34 extends beyond the distal end 38 of the post 30 to form the recess 40, and the implant 24/44/54 is retained within the recess 40. The physician determines the desired location for placement of the implant 24/44/54 and presses the sheath 34 against the tissue T at that desired location. Retraction of the sheath 34 toward the physician (e.g., backwards into the handle 32) exposes the implant 24/44/54 for seating within the tissue T.

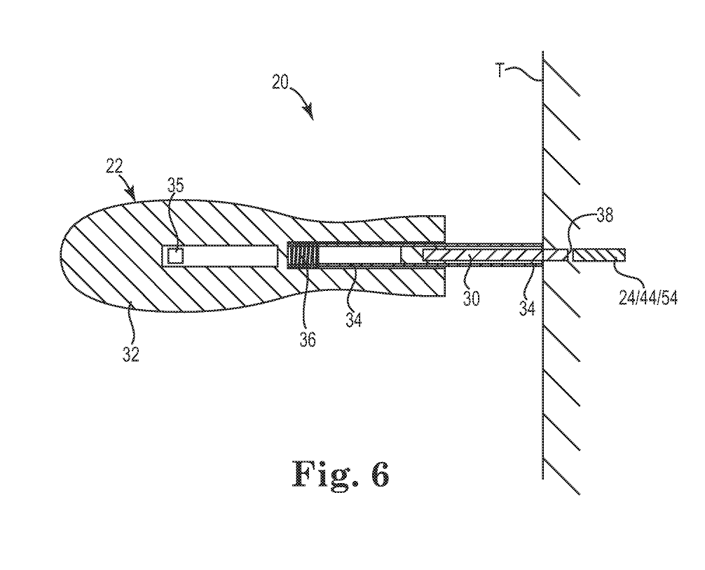

FIG. 6 is a cross-sectional view of the incontinence treatment system 20 in a deployed state. Pressing the sheath 34 against the tissue T forces the implant 24/44/54 into the tissue, and the sheath 34 is movable into the handle 32 to ease the implant 24/44/54 into place in a controlled manner. Thus, the implant 24/44/54 is gently guided into place and not shot or thrown into place. The button 35 is movable such that retracting the button 35 backward toward the physician will retract the sheath 34 into the handle 32, which allows the post 30 to be controllably guided forward to drive the implant 24/44/54 into the tissue T at the desired and pre-determined location relative to the patient. The implant 24/44/54 and the sheath 34 will encounter resistance as they interact with the tissue T to provide the physician with a sense of the depth that the implant 24/44/54 is pushed into the tissue T.

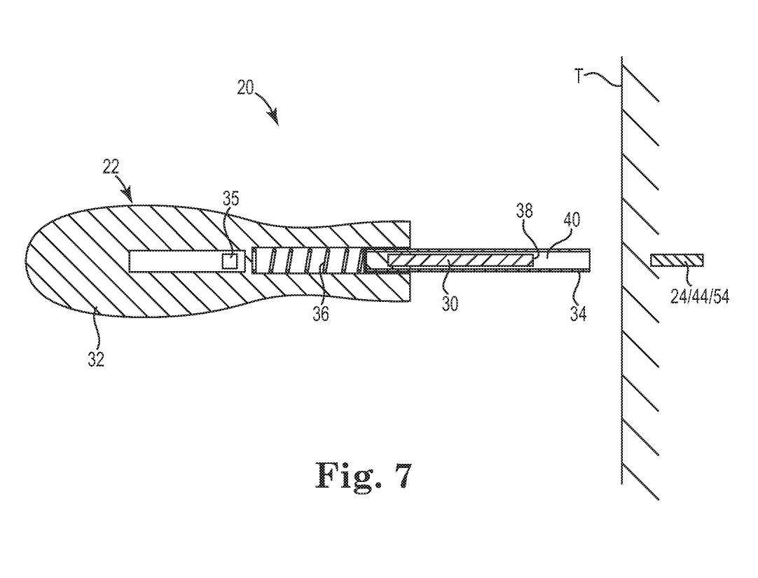

FIG. 7 is a cross-sectional view of the implant 24/44/54 injected into the tissue T with the insertion tool removed from the surface of the tissue to allow the sheath 34 to recover and extend over the post 30.

The post 30 and the sheath 34 are illustrated as straight and linear components. However, in one embodiment the post 30 and the sheath 34 are curved to allow the physician to position the implant 24/44/54 at a lateral distance was from the desired peri-urethral target location, and with the forward driving motion as aided by the curved post30/sheath 34, the physician delivers the implant 24/44/54 along an outside-to-inside path that places the implant 24/44/54 peri-urethrally at a position that is substantially parallel to the urethra.

In one embodiment, the method of treating incontinence to the use of the incontinence treatment system 20 includes inserting an implant, such as one of the implants 24/44/54 peri-urethrally into a patient until a longitudinal axis of the implant is aligned substantially parallel to a urethra of the patient. The implant occupies the peri-urethral tissue to limit the mobility of the urethra.

In one embodiment, a method of stabilizing a urethra in treating incontinence is provided that includes pushing an implant, such as one of the implants 24/44/54, peri-urethrally into a patient between submicosa tissue and smooth muscle tissue. The implant occupies a portion of the peri-urethral tissue to limit mobility of the urethra.

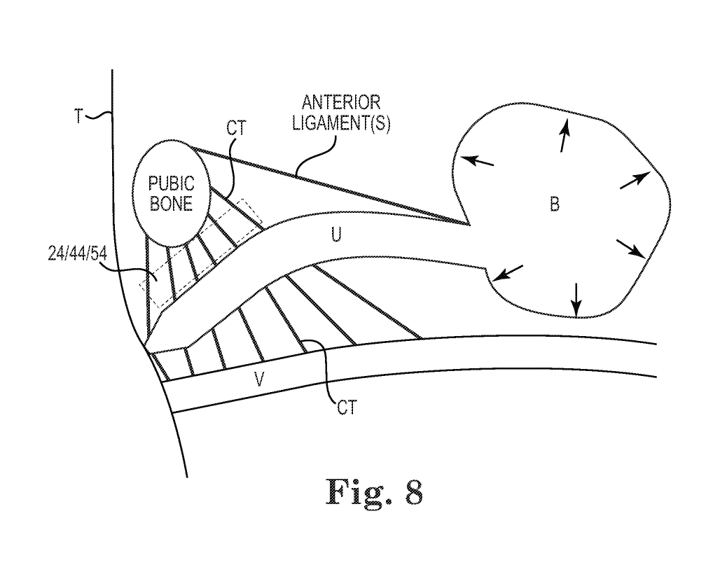

FIG. 8 is a schematic view of female anatomy including one embodiment of a female urethra U supported by one of the implants 24/44/54. The female anatomy includes the bladder B provided with the urethral urinary tract (or urethra U), the vagina V located inferior to the urethra U, and the pubic bone located superior to the urethra U. Connective tissue CT is connected between the urethra U and the pubic bone and the vagina V and pubic bone. Anterior ligaments connect and support the bladder relative to the pubic bone. In one embodiment, a single one of the implants 24/44/54 is implanted peri-urethrally into tissue (e.g., into peri-urethral tissue) that is remote from the urethra U and superior to the urethra. The implant 24/44/54 supports the urethra U, limiting mobility of the urethra U relative to the connective tissue CT and the vagina V, which assists the urethra U in maintaining continence.

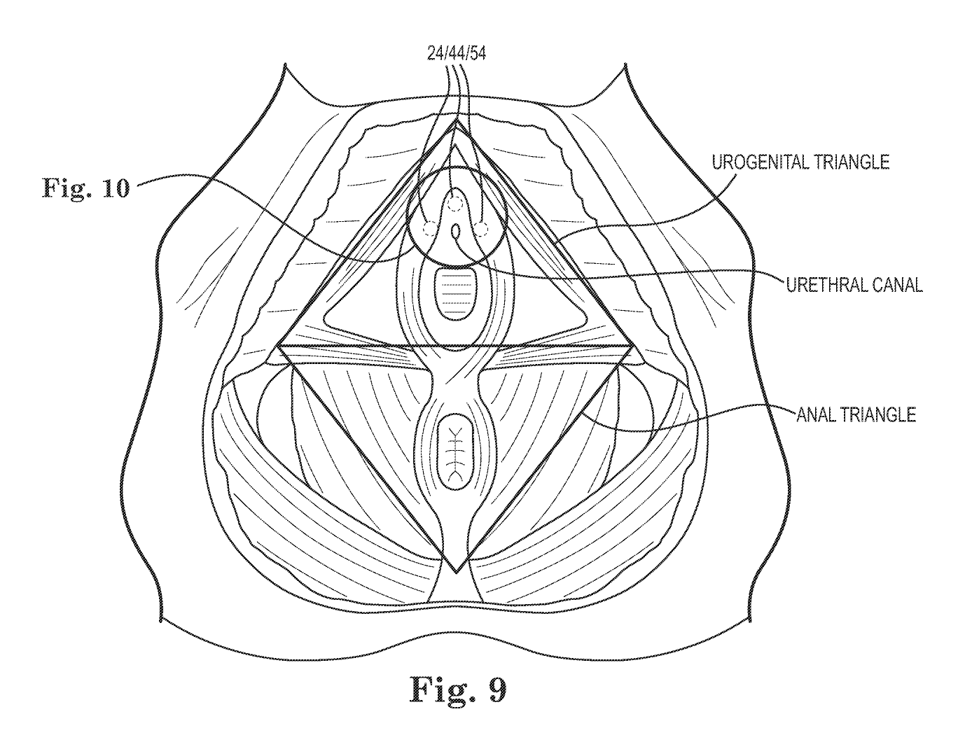

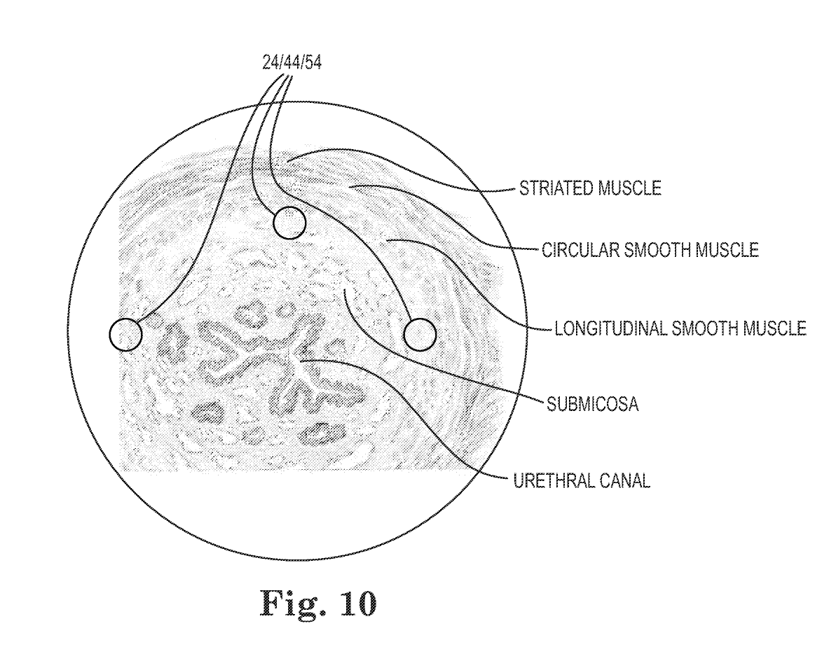

FIG. 9 is a schematic view of one embodiment of a female urethra supported by multiple implants 24/44/54 and FIG. 10 is a cross-sectional view of the female urethra and showing the implants 24/44/54. In this illustrated embodiment two of the implants 24/44/54 are implanted peri-urethrally lateral of the urethral canal and one of the implants 24/44/54 is implanted peri-urethrally superior to the urethral canal. In practice, the physician locates a peri-urethral target on an exterior surface of the tissue within the urogenital triangle, where the peri-urethral target is located generally between the urethral canal and the bulb of vestibule. The physician deploys the incontinence treatment system 20 (FIG. 5) as described above to insert the implants 24/44/54 one at a time within the peri-urethral target. The urethra is stabilized by locating the implant 24/44/54 peri-urethrally between submicosa tissue and smooth muscle tissue.

The implants 24/44/54 are implanted without the use of an incision, in part due to the structure of the implants 24/44/54 which enables the implants 24/44/54 to pierce the tissue when ejected from the insertion tool 22 (FIG. 5). Without being bound to this theory, it is believed that the implants 24/44/54 toughen the peri-urethral tissue, which results in the peri-urethral tissue providing increased support to the urethral canal to reduce or eliminate hyper-mobility of the urethra. In one embodiment, the implants 24/44/54 include an agent that induced fibrosis, and the fibrosis that is induced in the tissue contributes to the toughening of the peri-urethral tissue and support of the urethra.

Although specific embodiments have been illustrated and described herein, it will be appreciated by those of ordinary skill in the art that a variety of alternate and/or equivalent implementations may be substituted for the specific embodiments shown and described without departing from the scope of the present invention.

This application is intended to cover any adaptations or variations of medical devices as discussed herein. Therefore, it is intended that this invention be limited only by the claims and the equivalents thereof.

What is claimed is:

1. An incontinence treatment system comprising:
a rigid implant having an aspect ratio of greater than 5; and
an insertion tool comprising a post fixed to a handle and a sheath disposed around the post, the sheath axially retractable relative to the post and having a length that is greater than a length of the post;
wherein the insertion tool has a loaded state in which the sheath extends distal the post to provide a recess at a distal end of the post that is sized to receive the rigid implant and a deployed state in which the sheath retracts in a proximal direction toward the handle to expose the distal end of the post and allow the post to push the rigid implant out of the recess and into tissue.

2. The incontinence treatment system of claim 1, wherein the rigid implant comprises a silk rod.

3. The incontinence treatment system of claim 1, wherein the rigid implant comprises a polyester rod.

4. The incontinence treatment system of claim 1, wherein the rigid implant comprises an agent selected to induce a fibrotic response in the tissue.

5. The incontinence treatment system of claim 1, wherein the rigid implant has a longitudinal axis and is fabricated to include a pre-stressed bend such that one end of the rigid implant diverges away from the longitudinal axis when the body implantable rod is deployed in the tissue.

8. The incontinence treatment system of claim 6, wherein the implant comprises a rod-shaped implant.

9. An incontinence treatment system comprising:
a body implantable rod;
an insertion tool comprising a post fixed to a handle and a sheath disposed around the post, the sheath axially retractable relative to the post and having a length that is greater than a length of the post;
means for placing the body implantable rod peri-urethrally apart from a urethra of a patient and limiting mobility of the urethra.

10. The incontinence treatment system of claim 9, wherein the body implantable rod has a longitudinal axis and is fabricated to include a pre-stressed bend such that one end of the body implantable rod diverges away from the longitudinal axis when the body implantable rod is deployed in tissue.

11. The incontinence treatment system of claim 9, wherein the body implantable rod has a pre-stressed bend that is bendable such that the body implantable rod is constrained in a linear configuration when engaged in the insertion tool.
